(19) United States
(12) Patent Application Publication
(10) Pub. No.: US 2015/0305914 A1
(43) Pub. Date: Oct. 29, 2015

(54) MULTI-LAYER CAST SYSTEMS AND METHODS

(71) Applicant: THE REGENTS OF THE UNIVERSITY OF CALIFORNIA, Oakland, CA (US)

(72) Inventors: Benjamin M. Wu, San Marino, CA (US); Jeffrey C. Wang, Sherman Oaks, CA (US); Emily W. Loughran, Los Angeles, CA (US)

(73) Assignee: THE REGENTS OF THE UNIVERSITY OF CALIFORNIA, Oakland, CA (US)

(21) Appl. No.: 14/700,459

(22) Filed: Apr. 30, 2015

Related U.S. Application Data
(63) Continuation of application No. PCT/US2013/068180, filed on Nov. 2, 2013.
(60) Provisional application No. 61/722,164, filed on Nov. 3, 2012.

(51) Int. Cl. A61F 5/058; A61L 15/12

(52) U.S. Cl. CPC A61F 5/05841; A61L 15/12; A61F 2240/00

(57) ABSTRACT

A multi-layer cast for immobilizing a body member of a patient includes a hydrophobic sleeve having a shape configured to be applied over the body member and substantially conform to the shape of the body member, and a moldable layer configured to be positioned around said sleeve and hardened to conform to the shape of said body member adjacent said sleeve is presented. The hydrophobic sleeve includes fibers configured to wick moisture outward away from the body member, and the moldable layer includes a network of pores that extend through the moldable layer such that the pores are configured to contact the hydrophobic sleeve to promote flow of moisture from the hydrophobic sleeve outward through the moldable layer. An external removable wrap may also be detachably disposed around the moldable layer for additional support and moisture wicking.

### CROSS-REFERENCE TO RELATED APPLICATIONS

This application is a 35 U.S.C. §111(a) continuation of PCT international application number PCT/US2013/068180 filed on Nov. 2, 2013, incorporated herein by reference in its entirety, which claims priority to, and the benefit of, U.S. provisional patent application Ser. No. 61/722,164 filed on Nov. 3, 2012, incorporated herein by reference in its entirety. Priority is claimed to each of the foregoing applications.

The above-referenced PCT international application was published as PCT International Publication No. WO 2014/071265 on May 8, 2014, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to systems and methods to restrain anatomy, and more particularly to anatomical casting systems and methods.

2. Description of Related Art

Traditional casts are heavy, uncomfortable, and subject to molding and degradation by water and sweat. This often makes bathing, swimming and other activities difficult and/or impossible for the wearer. Further difficulties may be experienced in the ability for casts in adjusting to accommodate for swelling. The skin underneath the cast is also subject to irritation, skin breakdown, and pressure points that can lead to infections that can threaten the limb. The technology used in the casting process has changed little in 100 years. In addition, there are few truly waterproof casts that allow for fracture support along with the ability to submerge and wash the cast material once it is applied.

Accordingly, an object of the present invention is a light weight, supportive, porous, breathable and quick to dry cast that would overcome the majority of problems observed with plaster and fiberglass casts, and also allow for bathing and washing around the cast for general hygiene. At least some of these objectives will be met in the description below.

### BRIEF SUMMARY OF THE INVENTION

An aspect of the invention is a lightweight, supportive, porous, breathable and quick to dry cast having a plurality of layers configured to wick moisture away from the skin to allow for bathing and washing around the cast for general hygiene.

Another aspect is a multi-layer cast that immobilizes the body member of a patient, using a hydrophobic sleeve that has a shape configured to be applied over the body member and substantially conform to the shape of the body member. In one embodiment, the hydrophobic sleeve is made of fibers configured to wick moisture outward away from the body member. A moldable layer is configured to be positioned around the sleeve and hardened to conform to the shape of the body member adjacent to the sleeve. The moldable layer is comprised of a network of pores that extend through the moldable layer such that the pores are configured to contact the hydrophobic sleeve to promote flow of moisture from the hydrophobic sleeve outward through the moldable layer.

In a preferred embodiment, the multi-layer cast includes a removable external wrap configured to be detachably disposed around the moldable layer.

In a further embodiment, the hydrophobic sleeve is substantially non-hydrophilic and the external removable wrap is substantially hydrophilic, such that the moldable layer has a hydrophilicity value higher than the hydrophobic sleeve, but lower than the external removable wrap. The hydrophobic sleeve may also include a multi-layer structure comprising a hydrophobic inner layer and a hydrophilic outer layer.

Another aspect is a method of immobilizing a body member, where a hydrophobic sleeve is positioned over the body member and configured to substantially conform to the shape of the body member. The hydrophobic sleeve includes fibers configured to wick moisture outward away from the body member. A moldable layer is applied over the sleeve; and the moldable layer is then hardened to conform to the shape of the body member that is adjacent to the sleeve. The moldable layer included a network of pores that extend through the moldable layer such that the pores are configured to contact the hydrophilic sleeve to promote flow of moisture from the hydrophobic sleeve outward through the moldable layer.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 shows a cast system of the present invention disposed around the forearm of a patient.

FIG. 2 is a cross-sectional view of the three-layer cast system of FIG. 1.

FIG. 3 illustrates a close-up view of the sleeve of FIG. 1

FIG. 4 shows a cross-sectional view of the sleeve fibers of FIG. 1.

FIG. 5 illustrates a cross-sectional view of an alternative embodiment of the present invention comprising a two-layer cast system.

FIG. 6 illustrates an exploded view of the cast system of FIG. 1 and FIG. 2.

FIG. 7 shows a composite sleeve layer having multiple sub-layers.

FIG. 8 shows a flow diagram for immobilizing an anatomical feature according the invention.

### DETAILED DESCRIPTION OF THE INVENTION

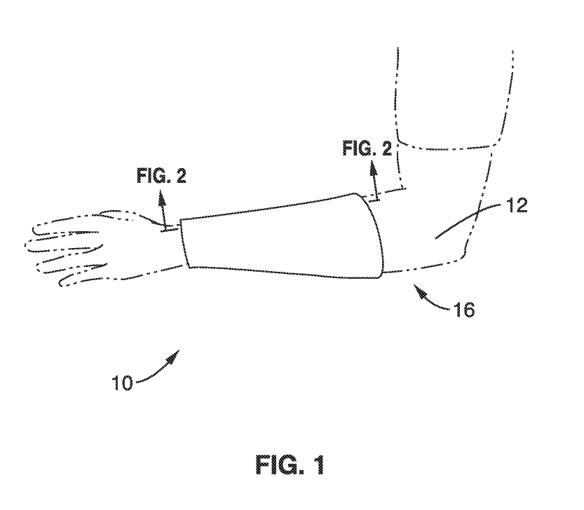

FIG. 1 shows a cast system 10 of the invention disposed around the skin surface 12 of a patient's forearm 16. It is appreciated that the embodiment shown in FIG. 1 may be used for immobilization of the forearm 16, e.g. for a fracture of the ulna or radius. It is further appreciated that cast system 10 may be used in a number of anatomical locations over the body of the patient to immobilize an anatomical region of interest (e.g. finger, wrist, foot, ankle, leg, etc.) that has sustained a fracture or soft tissue damage, or for other reasons where immobilization of the anatomy is desired by the physician.

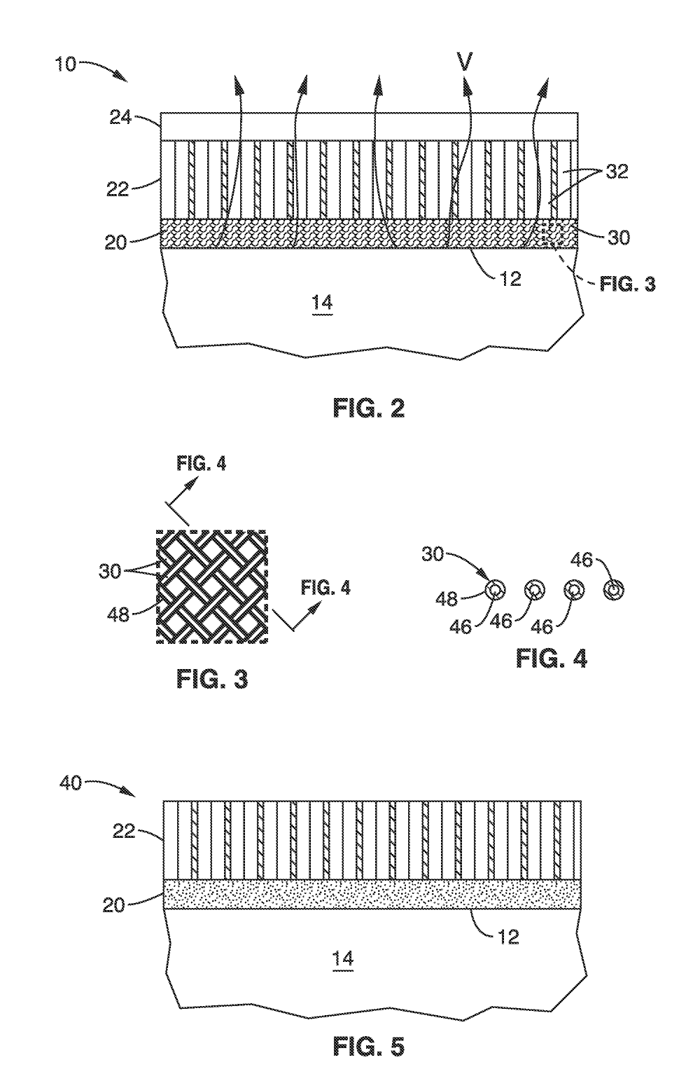

FIG. 2 is a cross-sectional view of the three-layer cast system 10 of FIG. 1 as disposed on the surface 12 of the patient's derma 14. The multi-layer cast system 10 is particularly configured for wicking moisture vapor V away from skin surface 12.

Cast system 10 comprises a first layer configured to contact and conform to skin surface 12 in the form of an elastic sleeve 20 comprising a fabric material made up of a network of wicking fibers 30 (see FIG. 3 and FIG. 4).

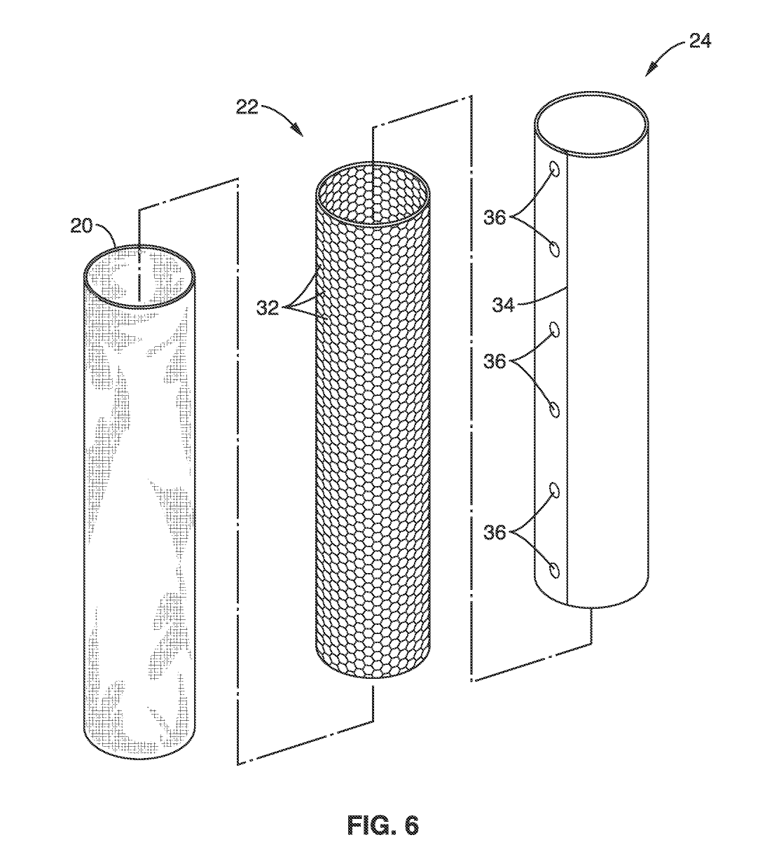

Disposed above sleeve 20 is a moldable layer 22 with a pattern of fine-gauge pores 32 (e.g. in a honeycomb pattern shown in FIG. 6). Layer 22 is a rigid, lightweight, moisture wicking layer that is configured is mold to the contours of the patient's skin 12 once set, to provide stability to the anatomy of interest.

An external removable wrap 24 comprises the third layer that is placed over the top of the second layer 22, and mainly functions as a super-wicking device to help draw moisture up from the two bottom layers 20 and 22.

FIG. 5 illustrates a cross-sectional view of an alternative embodiment of the present invention comprising a two-layer cast system 40. In this configuration, only the first sleeve layer 20 and moldable layer 22 are used, without a third or detachable layer. This configuration may be used in appropriate applications to provide moderate wicking and stability.

It is also appreciated that the properties of the sleeve layer 20 and moldable layer 22 may be combined to form a configuration having a single, moldable, lightweight, moisture wicking layer that may be hardened to rigidly support and immobilize the anatomy of interest.

FIG. 6 illustrates an exploded view of the cast system 10 of FIG. 1 and FIG. 2. Sleeve 20 is shown in FIG. 6 as a constant diameter tube. However, it is appreciated that the shape and diameter of sleeve 20 may vary across its length to more closely fit the patient's anatomy. Sleeve 20 may also comprise a slot or hole (not shown) to accommodate the patient's anatomy (e.g. thumb, etc.).

Rigid layer 22 may be molded to any tubular shape, e.g. cylindrical, frustro-conical, L-shaped, etc. The pores 32 may comprise a honeycomb pattern (as illustrated in FIG. 6), or other shapes as desired.

Removable outer layer 24 may comprise a seam or slit 34 to allow for easy detachment or attachment as needed. A plurality of snaps 36 or other fastening means may be used to hold seam 34 and outer layer 24 in place when disposed on the patient.

The sleeve 20 preferably comprises a hydrophobic material with high water vapor transfer rate (e.g. >800 g/m² per 24 hr). Sleeve 20 is preferably seamless, and may be provided in a kit that comes in a variety of sizes that fit snuggly over the limb 16 to provide both support for the limb and protection for the skin from irritation. The smooth snug fit of the sleeve 20 would also prevent any undesired pressure points that could lead to skin 12 breakdown and wound issues.

In a preferred embodiment, the sleeve 20 is constructed of a very light-weight soft synthetic material that wicks away moisture. A variety of moisture wicking fabrics available in the art may be used. The porous sleeve 20 comprises fibers 30 that may comprise a single fiber material, a mixture of fiber materials, or a functionally graded composite comprised of a multitude of fiber materials. As shown in FIG. 4, each of the fibers 30 may also comprise a composite construction comprising a core 46 of a first material and coating 48 of a second material.

For moisture wicking, fibers 30 are preferably hydrophobic and water repellent with minimal hygroscopicity, such that the moisture would not be retained in the fabric, and thus prolong drying time. Examples of hydrophobic, non-hygroscopic materials include polytetrafluoroethylene (PTFE, Gore-Tex) and other fluoropolymer fibers, polyethylene, polpropylene, and polyvinyl chloride.

In some instances, a limited amount of moisture retention may be desired. In such cases, a mixture of hydrophobic fibers with slight hygroscopicity (not shown) may be added to the aforementioned hydrophobic polymers to produce a slightly moist layer. Examples of hydrophobic fibers with slight hygroscopicity include polyesters (Rayon, Nylon, MicroTherm, DuoFold, DyrFit), polyurethane-polyurea (Spandex), bamboo, polyacrylonitriles, etc. In some cases, a small amount (<10%) of hydrophilic fibers (wool, cellulose, hydrophilic polyesters, acrylate viscose, rayon, etc.) may be added to increase moisture content.

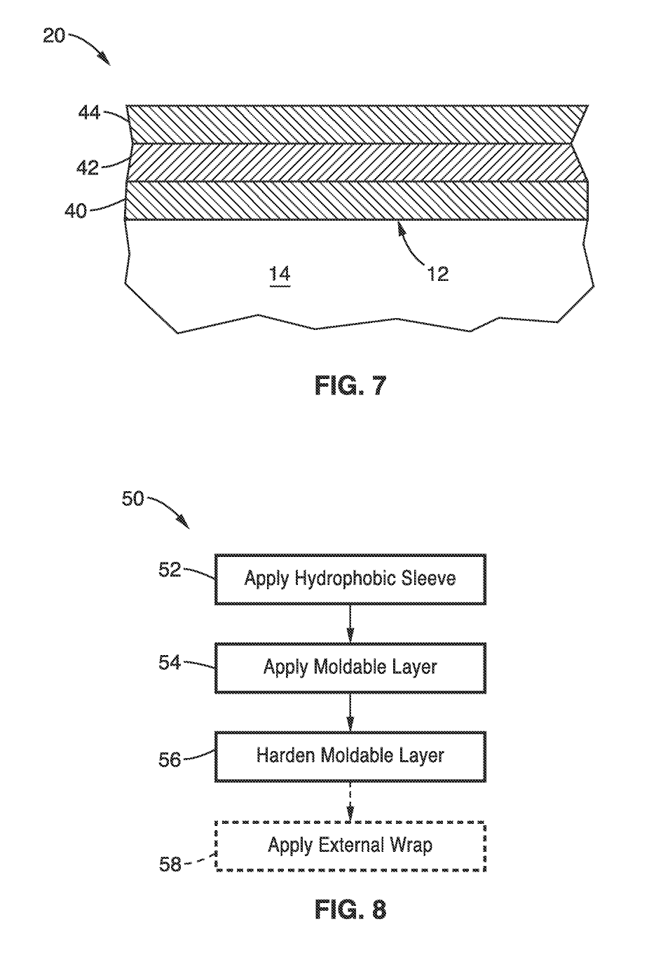

If greater moisture removal rate is needed, the sleeve 20 may be composed of a functionally graded composite layer that actively transports water away from the skin 12. This can be accomplished by strategically placing fiber layers to decrease the water contact angle from the skin 12 outwards toward the intermediate moldable layer 22. FIG. 7 shows a composite sleeve layer 20 having multiple sub-layers: a first inner layer 40 having hydrophobic, non-hygroscopic fibers on the skin side with micropores to pull liquid into the capillary systems; a second or middle layer 42 with fibers that are hydrophobic and slightly hygroscopic; and a third, outer layer 44 having hydrophobic, non-hygroscopic fibers with a hydrophilic coating 48 that pulls water to the outer-most surface for evaporation. In this configuration, the hydrophobic core 46 is constructed of a material (e.g. hydrophilic polyurethanes, silicones, etc.) that does not imbibe water.

In a preferred embodiment, the fibers 30 of the sleeve 20 may include a coating 48 comprising antimicrobial element to further protect the skin 12 from infection and irritation and prevent molding, which is a problem when casts accumulate sweat. If antimicrobial agents are added, the coating material 48 and the antimicrobial agents (e.g. silver, chitosan, etc.) will be selected based on the hydrophobic/hygroscopic guidelines described above.

As explained above, moldable middle layer 22 is configured to be altered after initial shape setting. Moldable layer 22 provides 3-point compression and stability to promote fracture healing that is molded over the limb 16 and the hydrophobic sleeve 20, and then hardened to secure the limb 16 and bone in the desired alignment. The porous configuration of moldable middle layer 22 forms a lightweight structure that provides the necessary three point pressure for bone healing under mild mechanical loading.

Moldable layer 22 comprises a network or pattern of fine gauge pores 32. In a preferred embodiment, pores 32 comprise a fine-gauge honeycomb pattern 32 shown in FIG. 6 that allows for airflow to the sleeve 20 so that any moisture or moisture vapor V from sweat or external sources flow from the sleeve layer 20 up through the second layer 22 of hydrophobic material. The porous pattern 32 would not irritate the skin 12, which is protected by the first layer 20.

Since layer 22 rests outside of the hydrophobic sleeve 20, the surface of layer 22 (which may comprise a coating) is preferably slightly more hydrophilic than then outer surface of the inner layer 20, such that moisture is transported away from the skin 12.

FIG. 8 shows a flow diagram for a method 50 of immobilizing an anatomical feature using the multi-layer cast 10 of the present invention.

First the hydrophobic sleeve 20 is positioned over the limb 16 of interest at step 52. The sleeve 20 is generally comprised of an elastic material that allows the sleeve 20 to be stretched as it is positioned to the anatomy of interest, and provide a small amount of compression to the skin surface and conform to the contours of the skin surface.

At step 54, the moldable layer 22 is then applied over the sleeve 22. In one embodiment, moldable layer 22 may be formed by extruding a hydrophobic polymeric layer with the pores 32 in one direction to generate a planar sheet having the pores running through a desired thickness (which may vary depending on application and anatomy to be immobilized). During application, the sheet may be rolled around the limb 14 until opposite ends touch or overlap, and excess material is cut away.

Next, at step 56, the moldable layer 22 is hardened to form a rigid structure around the sleeve 20. In one embodiment, the hardening of moldable layer 22 is accomplished by light activation. For instance, a polymer mesh, knitted from hollow fibers of transparent polypropylene (or polyethylene, etc) containing light activated monomer (bisGMA, TEGDMA, UDMA, etc.), photoinitiator, accelerator, inhibitor, etc. can be shaped around the broken limb 16 at step 54, and then a visible blue light is illuminated onto the mesh at step 56 to cause polymerization, rigidifying layer 22 to lock the bones and/or other tissues in alignment.

In the preferred embodiment, the moldable layer 22 may comprise a light-activated material such as Ultrabond Plus Resin Cement from Denmat Holdings, LLC, Lompoc, Calif. Such resin cement comprises glass fillers in methacrylate resin, silanated DMA, aluminum oxalate phosphoric acid, and citric acid solution. It is appreciated that for the purposes of this invention, the moldable layer 22 material may comprise one of more of the above constituent parts, or variants thereof, in order to form a preferred configuration in the particular use as a moldable cast component.

In one embodiment, the resin cement material for moldable layer 22 may be fabricated into a porous structure (e.g. honeycomb structure 32 (FIG. 2) via a mold, extraction, etc.) or woven into a porous mesh, so as to allow it to be applied or wrapped on the skin in a dry state, and then irradiated with blue light for hardening.

In another embodiment, the resin cement material for moldable layer 22 may be applied in the wet state as a thin coating to cover a pre-exiting mesh or porous bendable structure, and then irradiated with blue to harden the mesh or structure.

It is also contemplated that the moldable layer 22 may be composed of, but is not limited to, one or more of the following photopolymerizable polymers: acrylated polyurethane, methacrylated glycol chitosan, polyethylene oxide methacrylate, polyethylene oxide dimethacrylate, triethylene glycol dimethacrylate, urethane dimethacrylate, poly(propylene fumarate co-ethylene glycol), acrylated PVA, methacrylated, dextran, cinnamated hyaluronic acid, etc.

One or more of the following agents may be used to modify polymers to make them photopolymerizable: acrylate, methacrylate, dimethacrylate, cinnamate, thiol-ene, vinyl, or the like.

In an alternative embodiment, moldable layer 22 may be in the form of a thermoplastic mesh that allows adjustment of size and shape by first heating the layer locally with a controlled heat applicator prior to application step 54. Selection of this approach is limited by the melting temperature of the polymer, which should be low enough that the hydrophobic sleeve 20 and the underlying skin 12 are not affected. An example of a low temperature polymer is polycaprolactone (PCL), PCL-blends with polyurethanes, or other polyesters, which are more hydrophobic and stronger than the cotton based low temperature thermoplastic mesh casts that are currently used in the art. Other exemplary thermoplastic polymers include: thermoplastic polyurethane (various forms Tg ˜40-450), polybutylene terephthalate (Tg 40 C), polyvinylidene chloride (Tg 40 C), modifications to polymers to make them photopolymerizable: acrylate, methacrylate, dimethacrylate, cinnamate, thiol-ene, vinyl, etc.

In this configuration, the hardening step 56 is achieved via active or passive cooling of the layer 22. One advantage of a thermoplastic mesh is that excesses can be cut off at any location prior to setting.

As with sleeve layer 20, moldable layer 22 may also include antimicrobial agents. If antimicrobial agents are added, the coating material and the antimicrobial agents (e.g. silver, chitosan, etc.) may be selected based on the hydrophobic/hygroscopic guidelines described above.

At step 58, an optional third outer layer in the form of an external removable wrap 24 may then by applied over the hardened moldable layer 22.

External removable wrap 24 acts as a super wicking device to help draw moisture up from the two bottom layers 20, 22. The external removable wrap 24 could be interchangeably replaced as needed, and may include snaps 36 or like fasteners that allow the layer 24 to be removed prior to bathing or swimming. The primary requirement for this layer is strength, and many materials are available. Coating of external removable wrap 24 may be performed based on the hygroscopicity gradient described above for sleeve layer 20. External removable wrap 24 is preferably the most hydrophilic and non-hygroscopic of the three-layer cast 10 in order to facilitate moisture evaporation.

Experimental Results:

In one embodiment, TEGDMA and CQ with blue light photopolymerization were tested as a moldable layer 22. The constituent parts comprised 95% TEGDMA from stock, 0.2% CQ w/v, 100 ul on glass slide, and were irradiated for 30 s or 60 s with VER3-66 or Bisco VIP 6×100 mW/cm². In other embodiments, various concentrations of TEGDMA and CQ were also tested: e.g. TEGDMA at 100%, 50%, or 1% v/v, and CQ at 20%, 10%, or 1% w/v and irradiated in 30 s increments, e.g. for 30 s, 60 s 90 s, and up to 3 min. Various degrees of polymerization were achieved.

In another embodiment, UEDMA and CQ with blue light photopolymerization were tested as a moldable layer 22. The constituent parts comprised 80% UEDMA from stock, 0.2% CQ 0.2% w/v, 100 ul on glass slide, and were irradiated for 30 s or 60 s with VER3-66 or Bisco VIP 6×100 mW/cm².

From the discussion above it will be appreciated that the invention can be embodied in various ways, including the following:

1. A multi-layer cast for immobilizing a body member of a patient, comprising: a hydrophobic sleeve having a shape configured to be applied over the body member and substantially conform to the shape of the body member; and a moldable layer configured to be positioned around said sleeve and hardened to conform to the shape of said body member adjacent said sleeve; the moldable layer comprising a network of pores that extend through the moldable layer; wherein the pores are configured to contact the hydrophobic sleeve to promote flow of moisture from the hydrophobic sleeve outward through the moldable layer.

2. A multi-layer cast as in any of the previous embodiments, wherein the hydrophobic sleeve comprises fibers configured to wick moisture outward away from the body member.

3. A multi-layer cast as in any of the previous embodiments, further comprising: an external wrap configured to be disposed around the moldable layer.

4. A multi-layer cast as in any of the previous embodiments: wherein the hydrophobic sleeve is substantially non-hydrophilic and wherein the external removable wrap is substantially hydrophilic; wherein the moldable layer has a hydrophilicity value higher than the hydrophobic sleeve, but lower than the external removable wrap.

5. A multi-layer cast as in any of the previous embodiments: wherein the external wrap comprises a removable external wrap configured to be detachably disposed around the moldable layer.

6. A multi-layer cast as in any of the previous embodiments, wherein the hydrophobic sleeve comprises a multi-layer structure comprising a hydrophobic inner layer and a hydrophilic outer layer.

7. A multi-layer cast as in any of the previous embodiments, wherein the hydrophilic outer layer comprises fibers having hydrophobic core and hydrophilic coating.

8. A multi-layer cast as in any of the previous embodiments, wherein the moldable layer comprises a polymer that is hardened by light activation.

9. A multi-layer cast as in any of the previous embodiments, wherein the moldable layer comprises a thermoplastic.

10. A method of immobilizing a body member, comprising: positioning a hydrophobic sleeve over the body member; the hydrophobic sleeve having a shape configured to be applied over the body member and substantially conform to the shape of the body member; applying a moldable layer over the sleeve; and hardening the moldable layer to conform to the shape of said body member adjacent said sleeve; wherein the moldable layer comprises a network of pores that extend through the moldable layer; wherein the pores are configured to contact the hydrophobic sleeve to promote flow of moisture from the hydrophobic sleeve outward through the moldable layer.

11. A method as in any of the previous embodiments, wherein the hydrophobic sleeve comprises fibers configured to wick moisture outward away from the body member.

12. A method as in any of the previous embodiments, further comprising: disposing an external wrap around the moldable layer.

13. A method as in any of the previous embodiments: wherein the hydrophobic sleeve is substantially non-hydrophilic and wherein the external removable wrap is substantially hydrophilic; and wherein the moldable layer has a hydrophilicity value higher than the hydrophobic sleeve, but lower than the external removable wrap.

14. A method as in any of the previous embodiments, wherein the external wrap is detachably disposed around the moldable layer.

15. A method as in any of the previous embodiments, wherein the hydrophobic sleeve comprises a multi-layer structure comprising a hydrophobic inner layer and a hydrophilic outer layer.

16. A method as in any of the previous embodiments, wherein the hydrophilic outer layer comprises fibers having hydrophobic core and hydrophilic coating.

17. A method as in any of the previous embodiments, wherein hardening the moldable layer comprises exposing the moldable layer to light.

18. A method as in any of the previous embodiments, wherein the moldable layer comprises a thermoplastic.

19. A cast for immobilizing a body member of a patient, comprising: a sleeve having a shape configured to be applied over the body member and substantially conform to the shape of the body member; a moldable layer configured to be positioned around said sleeve and hardened to conform to the shape of said body member adjacent said sleeve; the moldable layer comprising a network of pores; wherein the pores are configured to contact the hydrophobic sleeve to promote flow or evaporation of moisture from the hydrophobic sleeve outward through the moldable layer.

20. A cast as in any of the previous embodiments, wherein the sleeve comprises a hydrophobic sleeve having fibers configured to wick moisture outward away from the body member.

21. A cast as in any of the previous embodiments, further comprising: an external wrap configured to be disposed around the moldable layer.

22. A cast as in any of the previous embodiments: wherein the hydrophobic sleeve is substantially non-hydrophilic and wherein the external removable wrap is substantially hydrophilic; and wherein the moldable layer has a hydrophilicity value higher than the hydrophobic sleeve, but lower than the external removable wrap.

23. A cast as in any of the previous embodiments, wherein the external wrap comprises a removable external wrap configured to be detachably disposed around the moldable layer.

24. A cast as in any of the previous embodiments, wherein the hydrophobic sleeve comprises a multi-layer structure comprising a hydrophobic inner layer and a hydrophilic outer layer.

25. A cast as in any of the previous embodiments, wherein the hydrophilic outer layer comprises fibers having hydrophobic core and hydrophilic coating.

26. A cast for immobilizing a body member of a patient, comprising: a hydrophobic sleeve having a shape configured to be applied over the body member and substantially conform to the shape of the body member; a moldable layer configured to be positioned around the body member and hardened to conform to the shape of said body member; the moldable layer comprising a network of pores that extend through the moldable layer; wherein the pores are configured to contact the body member to promote flow of moisture from the body member outward through the moldable layer.

27. A cast as in any of the previous embodiments, wherein the moldable layer comprises Ultrabond Plus Resin Cement from Denmat Holdings.

28. A cast as in any of the previous embodiments, wherein the moldable layer comprises a resin cement comprising one or more of the following: glass fillers, methacrylate resin, silanated DMA, aluminum oxalate phosphoric acid, and citric acid solution.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A multi-layer cast for immobilizing a body member of a patient, comprising:
a hydrophobic sleeve having a shape configured to be applied over the body member and substantially conform to the shape of the body member; and
a moldable layer configured to be positioned around said sleeve and hardened to conform to the shape of said body member adjacent said sleeve;
the moldable layer comprising a network of pores that extend through the moldable layer;
wherein the pores are configured to contact the hydrophobic sleeve to promote flow of moisture from the hydrophobic sleeve outward through the moldable layer.

2. A multi-layer cast as recited in claim 1, wherein the hydrophobic sleeve comprises fibers configured to wick moisture outward away from the body member.

3. A multi-layer cast as recited in claim 1, further comprising:
an external wrap configured to be disposed around the moldable layer.

4. A multi-layer cast as recited in claim 3:
wherein the hydrophobic sleeve is substantially non-hydrophilic and wherein the external removable wrap is substantially hydrophilic; and
wherein the moldable layer has a hydrophilicity value higher than the hydrophobic sleeve, but lower than the external removable wrap.

5. A multi-layer cast as recited in claim 3, wherein the external wrap comprises a removable external wrap configured to be detachably disposed around the moldable layer.

6. A multi-layer cast as recited in claim 1, wherein the hydrophobic sleeve comprises a multi-layer structure comprising a hydrophobic inner layer and a hydrophilic outer layer.

7. A multi-layer cast as recited in claim 6, wherein the hydrophilic outer layer comprises fibers having hydrophobic core and hydrophilic coating.

8. A multi-layer cast as recited in claim 1, wherein the moldable layer comprises a polymer that is hardened by light activation.

9. A multi-layer cast as recited in claim 1, wherein the moldable layer comprises a thermoplastic.

10. A method of immobilizing a body member, comprising:
positioning a hydrophobic sleeve over the body member;
the hydrophobic sleeve having a shape configured to be applied over the body member and substantially conform to the shape of the body member;
applying a moldable layer over the sleeve; and
hardening the moldable layer to conform to the shape of said body member adjacent said sleeve;
wherein the moldable layer comprises a network of pores that extend through the moldable layer; and
wherein the pores are configured to contact the hydrophobic sleeve to promote flow of moisture from the hydrophobic sleeve outward through the moldable layer.

11. A method as recited in claim 10, wherein the hydrophobic sleeve comprises fibers configured to wick moisture outward away from the body member.

12. A method as recited in claim 10, further comprising:
disposing an external wrap around the moldable layer.

13. A method as recited in claim 12:
wherein the hydrophobic sleeve is substantially non-hydrophilic and wherein the external removable wrap is substantially hydrophilic; and
wherein the moldable layer has a hydrophilicity value higher than the hydrophobic sleeve, but lower than the external removable wrap.

14. A method as recited in claim 12, wherein the external wrap is detachably disposed around the moldable layer.

15. A method as recited in claim 10, wherein the hydrophobic sleeve comprises a multi-layer structure comprising a hydrophobic inner layer and a hydrophilic outer layer.

16. A method as recited in claim 15, wherein the hydrophilic outer layer comprises fibers having hydrophobic core and hydrophilic coating.

17. A method as recited in claim 10, wherein hardening the moldable layer comprises exposing the moldable layer to light.

18. A method as recited in claim 10, wherein the moldable layer comprises a thermoplastic.

19. A cast for immobilizing a body member of a patient, comprising:
a sleeve having a shape configured to be applied over the body member and substantially conform to the shape of the body member; and
a moldable layer configured to be positioned around said sleeve and hardened to conform to the shape of said body member adjacent said sleeve;
the moldable layer comprising a network of pores;
wherein the pores are configured to contact the sleeve to promote flow or evaporation of moisture from the sleeve outward through the moldable layer.

20. A cast as recited in claim 19, wherein the sleeve comprises a hydrophobic sleeve having fibers configured to wick moisture outward away from the body member.

21. A cast as recited in claim 20, further comprising:
an external wrap configured to be disposed around the moldable layer.

22. A cast as recited in claim 21:
wherein the hydrophobic sleeve is substantially non-hydrophilic and wherein the external removable wrap is substantially hydrophilic; and
wherein the moldable layer has a hydrophilicity value higher than the hydrophobic sleeve, but lower than the external removable wrap.

23. A cast as recited in claim 20, wherein the external wrap comprises a removable external wrap configured to be detachably disposed around the moldable layer.

24. A cast as recited in claim 20, wherein the hydrophobic sleeve comprises a multi-layer structure comprising a hydrophobic inner layer and a hydrophilic outer layer.

25. A cast as recited in claim 24, wherein the hydrophilic outer layer comprises fibers having hydrophobic core and hydrophilic coating.
